(19) United States
(12) Patent Application Publication
(10) Pub. No.: US 2015/0080420 A1
(43) Pub. Date: Mar. 19, 2015

(54) AMORPHOUS FORM OF LOPINAVIR AND RITONAVIR MIXTURE

(71) Applicant: HETERO RESEARCH FOUNDATION, Hyderabad (IN)

(72) Inventors: Bandi Parthasaradhi Reddy, Hyderabad (IN); Kura Rathnakar Reddy, Hyderabad (IN); Dasari Muralidhara Reddy, Hyderabad (IN); Kesireddy Subash Chander Reddy, Hyderabad (IN); Bandi Vamsi Krishna, Hyderabad (IN)

(21) Appl. No.: 14/319,755

(22) Filed: Jun. 30, 2014

Related U.S. Application Data
(63) Continuation of application No. 14/003,535, filed on Sep. 6, 2013, now abandoned.

(30) Foreign Application Priority Data
Mar. 7, 2011 (IN) 665/CHE/2011

(51) Int. Cl. A61K 31/513; A61K 31/427

(52) U.S. Cl. CPC A61K 31/513; A61K 31/427
USPC 514/274

(57) ABSTRACT

The present invention relates to a novel amorphous Form of lopinavir and ritonavir mixture in the ratio of 3.8:1.2 to 4.2:0.8, process for its preparation and pharmaceutical compositions comprising it.

### CROSS-REFERENCE TO RELATED APPLICATIONS

This application is a continuation of prior U.S. application Ser. No. 14/003,535 with a filing date of Mar. 5, 2012 which is a U.S. national stage of international application No. PCT/IN2012/000156, filed on Mar. 5, 2012, the disclosures of which are each incorporated herein by reference in their entirety. Priority is claimed from IN Patent Application No. 665/CHE/2011, filed Mar. 7, 2011, the disclosure of which is also incorporated herein by reference in its entirety.

### FIELD OF THE INVENTION

The present invention relates to a novel amorphous Form of lopinavir and ritonavir mixture in the ratio of 3.8:1.2 to 4.2:0.8, process for its preparation and pharmaceutical compositions comprising it.

### BACKGROUND OF THE INVENTION

Inhibitors of human immunodeficiency virus (HIV) protease have been approved for use in the treatment of HIV infection for several years. A particularly effective HIV protease inhibitor was (2S,3S,5S)-2-(2,6-dimethylphenoxyacetyl)amino-3-hydroxy-5-(2-1-tetrahydropyrimid-2-onyl)-3-methylbutanoyl)amino-1,6-diphenylhexane, also known as lopinavir.

Lopinavir was known to have ability of inhibiting HIV protease and the HIV infection. Lopinavir was particular effective for the inhibition of HIV protease and for the inhibition of HIV infection when co-administered with Ritonavir.

The combination of lopinavir and ritonavir is marketed in the dosage strength 133.3:33.3; 80:20; 100:25; and 200:50 under the brand name of KALETRA®.

Ritonavir was chemically, (5S,8S,10S,11S)-10-Hydroxy-2-methyl-5-(1-methylethyl)-1-[2-(1-methylethyl)-4-thiazolyl]-3,6-dioxo-8,11-bis(phenylmethyl)-2,4,7,12-tetraazatridecan-13-oic acid 5-thiazolylmethyl ester.

Lopinavir and its process were disclosed in U.S. Pat. No. 5,914,332. According to the patent, amorphous lopinavir can be prepared by dissolving lopinavir in a solvent such as absolute ethanol, isopropanol, acetone or acetonitrile and then adding the solution to water.

Ritonavir and its process were disclosed in U.S. Pat. No. 5,541,206.

Polymorphism is defined as “the ability of a substance to exist as two or more crystalline phases that have different arrangement and/or conformations of the molecules in the crystal Lattice. Thus, in the strict sense, polymorphs are different crystalline Forms of the same pure substance in which the molecules have different arrangements and/or different configurations of the molecules”. Different polymorphs may differ in their physical properties such as melting point, solubility, X-ray diffraction patterns, etc. Although those differences disappear once the compound is dissolved, they can appreciably influence pharmaceutically relevant properties of the solid form, such as handling properties, dissolution rate and stability. Such properties can significantly influence the processing, shelf life, and commercial acceptance of a polymorph. It is therefore important to investigate all solid forms of a drug, including all polymorphic forms, and to determine the stability, dissolution and flow properties of each polymorphic form. Polymorphic forms of a compound can be distinguished in the laboratory by analytical methods such as X-ray diffraction (XRD), Differential Scanning calorimetry (DSC) and Infrared spectrometry (IR).

Solvent medium and mode of crystallization play very important role in obtaining a crystalline Form over the other.

A mixture of lopinavir and ritonavir can exist in different polymorphic Forms, which may differ from each other in terms of stability, physical properties, spectral data and methods of preparation.

PCT Publication No. WO 2001/74787 described various polymorphic Forms of lopinavir and processes for their preparation. The Publication described the formation of several polymorphic Forms of lopinavir, which were designated lopinavir crystal Form of Type I hydrated, Type I higher hydrated, Type II isopropanol hemisolvate, Type II isopropanol solvate, Type II ethyl acetate hemisolvate, Type II ethyl acetate solvate, Type II chloroform hemisolvate, Type III ethyl acetate solvated, Type III de-solvated and Type IV non-solvated.

A process for the preparation of lopinavir amorphous Form was disclosed in PCT publication nos. WO 2009/004653 and WO 2009/019661.

PCT publication no. WO 2010/089753 disclosed a de-solvated crystalline Form H1 and cyclohexane solvate Form of lopinavir.

An unpublished application, IN 303/CHE/2011 assigned to Hetero research foundation discloses a process for the preparation of lopinavir amorphous Form, lopinavir de-solvated crystalline Form H2 and lopinavir de-solvated crystalline Form H3.

Crystalline Form II of ritonavir was disclosed in U.S. Pat. No. 6,894,171.

U.S. Pat. No. 7,205,413 disclosed crystalline Form III, Form IV and Form V of ritonavir.

U.S. Pat. No. 7,148,359 disclosed a substantially pure amorphous ritonavir.

We have surprisingly found that lopinavir and ritonavir mixture can be prepared in amorphous Form. The novel amorphous Form has been found to be stable over the time and reproducible and so, suitable for pharmaceutical preparations. The amorphous Form of lopinavir and ritonavir mixture obtained by the process of the invention may be used directly in development of combination composition of lopinavir and ritonavir.

Thus, an object of the present invention is to provide novel amorphous Form of lopinavir and ritonavir mixture in the ratio of 3.8:1.2 to 4.2:0.8, process for its preparation and pharmaceutical compositions comprising it.

### SUMMARY OF THE INVENTION

In one aspect, the present invention provides a novel amorphous Form of lopinavir and ritonavir mixture in the ratio of 3.8:1.2 to 4.2:0.8.

In another aspect, the present invention provides a process for the preparation of amorphous Form of lopinavir and ritonavir mixture in the ratio of 3.8:1.2 to 4.2:0.8, which comprises:

a) dissolving a mixture of lopinavir and ritonavir in an alcoholic solvent; and

b) removing the solvent by drying at about 60 to 80° C. to obtain amorphous Form of lopinavir and ritonavir mixture in the ratio of 3.8:1.2 to 4.2:0.8.

Yet another aspect, the present invention provides a pharmaceutical composition comprising amorphous Form of lopinavir and ritonavir mixture in the ratio of 3.8:1.2 to 4.2:0.8 and pharmaceutically acceptable excipients.

### BRIEF DESCRIPTION OF THE DRAWINGS

FIG. 1 is an X-ray powder diffraction spectrum of amorphous Form of lopinavir and ritonavir mixture in the ratio of 3.8:1.2 to 4.2:0.8.

X-ray powder diffraction spectrum was measured on a bruker axs D8 advance X-ray powder diffractometer having a copper-Kα radiation. Approximately 500 mg of sample was gently flattered on a sample holder and scanned from 2 to 50 degrees two-theta, at 0.019 degrees to theta per step and a step of 119 seconds. The sample was simply placed on the sample holder. The sample was rotated at 30 rpm at a voltage 40 KV and current 35 mA.

### DETAILED DESCRIPTION OF THE INVENTION

The term “room temperature” refers to temperature at about 25 to 35° C.

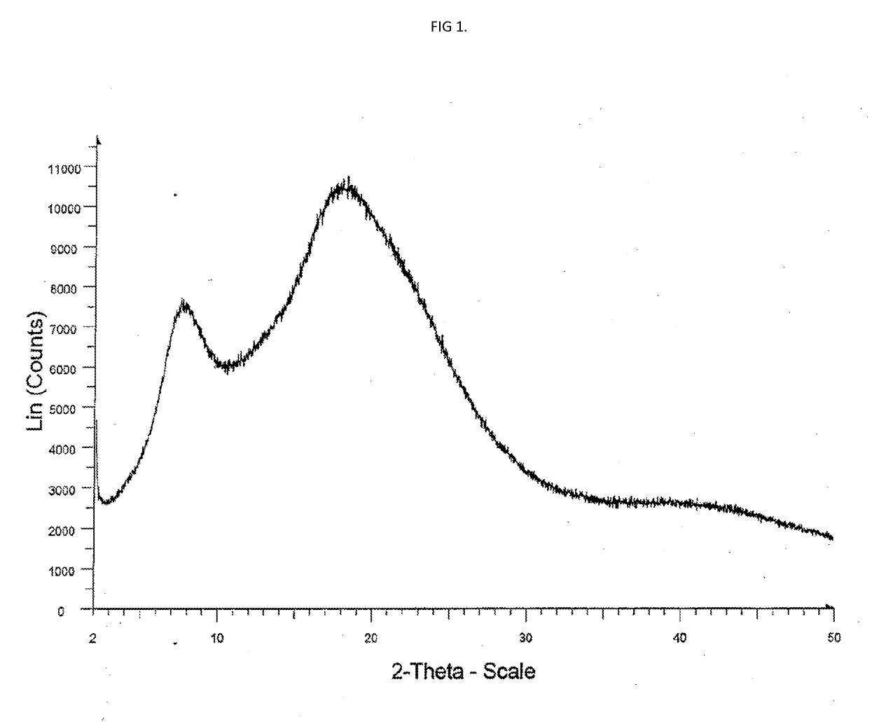

According to one aspect of the present invention, there is provided a novel amorphous Form of lopinavir and ritonavir mixture in the ratio of 3.8:1.2 to 4.2:0.8. The powdered x-ray diffractogram (PXRD) of amorphous Form of lopinavir and ritonavir mixture in the ratio of 3.8:1.2 to 4.2:0.8 is shown in FIG. 1.

According to another aspect of the present invention, there is provided a process for the preparation of amorphous Form of lopinavir and ritonavir mixture in the ratio of 3.8:1.2 to 4.2:0.8, which comprises:

a) dissolving a mixture of lopinavir and ritonavir in an alcoholic solvent; and

b) removing the solvent by drying at about 60 to 80° C. to obtain amorphous Form of lopinavir and ritonavir mixture in the ratio of 3.8:1.2 to 4.2:0.8.

Lopinavir and ritonavir used in step (a) may be any known crystalline or amorphous Forms.

The alcoholic solvent used in step (a) may preferably be a solvent or mixture of solvents selected from methanol, ethanol, isopropyl alcohol and n-butanol, and more preferably the alcoholic solvent is ethanol.

The dissolution in step (a) may be performed, for example, by heating the mixture of lopinavir and ritonavir in the solvent.

Drying in step (b) may preferably be carried out at about 65 to 75° C. under high vacuum.

According to another aspect of the present invention, there is provided a pharmaceutical composition comprising amorphous Form of lopinavir and ritonavir mixture in the ratio of 3.8:1.2 to 4.2:0.8 and pharmaceutically acceptable excipients, and optionally other therapeutic ingredients. The amorphous Form may preferably be formulated into tablets, capsules, suspensions, dispersions, injectables or other pharmaceutical forms.

The invention will now be further described by the following examples, which are illustrative rather than limiting.

### EXAMPLES

### Preparation of Amorphous Form of Lopinavir and Ritonavir Mixture in the Ratio of 4:1:

### Example 1

A mixture of lopinavir ethyl acetate solvate (400 gm) and ritonavir (100 gm) was dissolved in ethanol (1250 ml) under stirring at room temperature. The solution was then heated to 40 to 45° C. and then treated with carbon. The resulting solution was subjected to tray dried under high vacuum at 65 to 70° C. for 13 hours to obtain amorphous Form of lopinavir and ritonavir mixture in the ratio of 4:1.

### Example 2

A mixture of lopinavir cyclohexane solvate (40 gm) and ritonavir (10 gm) was dissolved in ethanol (120 ml) at room temperature. The solution was then heated to 40 to 45° C. and then treated with carbon. The resulting solution was dried under high vacuum at 65 to 70° C. for 12 hours to obtain amorphous Form of lopinavir and ritonavir mixture in the ratio of 4:1.

### Example 3

Example 1 was repeated using lopinavir de-solvated crystalline Form H1 instead of lopinavir ethyl acetate solvate to obtain amorphous Form of lopinavir and ritonavir mixture in the ratio of 4:1.

### Example 4

Example 1 was repeated using lopinavir de-solvated crystalline Form H2 instead of lopinavir ethyl acetate solvate to obtain amorphous Form of lopinavir and ritonavir mixture in the ratio of 4:1.

### Example 5

Example 1 was repeated using lopinavir de-solvated crystalline Form H3 instead of lopinavir ethyl acetate solvate to obtain amorphous Form of lopinavir and ritonavir mixture in the ratio of 4:1.

### Example 6

Example 2 was repeated using lopinavir type I hydrated instead of lopinavir cyclohexane solvate to obtain amorphous Form of lopinavir and ritonavir mixture in the ratio of 4:1.

### Example 7

Example 2 was repeated using lopinavir type I higher hydrated instead of lopinavir cyclohexane solvate to obtain amorphous Form of lopinavir and ritonavir mixture in the ratio of 4:1.

### Example 8

Example 1 was repeated using methanol solvent instead of ethanol solvent to obtain amorphous Form of lopinavir and ritonavir mixture in the ratio of 4:1.

### Example 9

Example 2 was repeated using isopropyl alcohol solvent instead of ethanol solvent to obtain amorphous Form of lopinavir and ritonavir mixture in the ratio of 4:1.

We claim:

1. Amorphous Form of lopinavir and ritonavir mixture in the ratio of 3.8:1.2 to 4.2:0.8.

2. Amorphous Form of lopinavir and ritonavir mixture in the ratio of 3.8:1.2 to 4.2:0.8, characterized by an x-ray powder diffractogram as shown in FIG. 1.

3. A process for the preparation of amorphous Form of lopinavir and ritonavir mixture in the ratio of 3.8:1.2 to 4.2:0.8, which comprises:
a. dissolving a mixture of lopinavir and ritonavir in an alcoholic solvent; and
b. removing the solvent by drying at about 60 to 80° C. to obtain amorphous Form of lopinavir and ritonavir mixture in the ratio of 3.8:1.2 to 4.2:0.8.

4. The process as claimed in claim 3, wherein the alcoholic solvent used in step (a) is a solvent or mixture of solvents selected from methanol, ethanol, isopropyl alcohol and n-butanol.

5. The process as claimed in claim 4, wherein the alcoholic solvent is ethanol.

6. A pharmaceutical composition that comprises amorphous Form of lopinavir and ritonavir mixture in the ratio of 3.8:1.2 to 4.2:0.8 and pharmaceutically acceptable excipients, and optionally other therapeutic ingredients.

7. The pharmaceutical composition as claimed in claim 6, wherein the amorphous Form is formulated into tablets, capsules, suspensions, dispersions or injectables.
